(19) United States
(12) Patent Application Publication
(10) Pub. No.: US 2024/0017036 A1
(43) Pub. Date: Jan. 18, 2024

(54) SCENT PROVISION APPARATUS AND SCENT PROVISION METHOD

(71) Applicant: SONY GROUP CORPORATION, TOKYO (JP)

(72) Inventors: SHUJI FUJITA, TOKYO (JP); YUKITO INOUE, TOKYO (JP); KAZUTAKA TAKAKI, TOKYO (JP)

(21) Appl. No.: 18/254,378

(22) PCT Filed: Oct. 13, 2021
(86) PCT No.: PCT/JP2021/037837

§ 371 Date: May 24, 2023

(30) Foreign Application Priority Data
Dec. 1, 2020 (JP) 2020-199598

(51) Int. Cl. A61M 21/00

(52) U.S. Cl. CPC A61M 21/00; A61M 2021/0016; A61M 2205/07

(57) ABSTRACT

To Provide a scent provision apparatus capable of providing a scent to a user at a proper timing and preventing the provision of a scent at an unintended timing. Provided is a scent provision apparatus 200 that includes a main body section 201 and an air flow control unit 203. The main body section 201 has an opening portion 207 for discharging or taking in a scent-containing air flow that contains a scent. The air flow control unit 203 controls characteristics of the scent-containing air flow discharged or taken in from the opening portion 207. The air flow control unit 203 can generate a control air flow for accelerating and decelerating and/or diffusing the scent-containing air flow and can also generate a control air flow for interrupting a flow of the scent-containing air flow.

### TECHNICAL FIELD

The present technology relates to a scent provision apparatus and a scent provision method and, more specifically, to a scent provision apparatus and a scent provision method for providing a scent to a user by an air flow that contains the scent.

### BACKGROUND ART

Although devices for presenting a scent to a user are known to date, it is important for such devices to present the scent to the user without giving a sense of discomfort. Therefore, there has been proposed a device that is capable of preventing presentation of an unintended scent and switching between scents to be presented.

For example, PTL 1 proposes a perfume presentation device that is used together with a display device wearable on a user's head and that includes a perfume presentation module, a perfume holder, and a perfume conductor. The perfume presentation module is arranged at a perfume presentation position in the vicinity of nostrils of a wearer of the display device. The perfume holder includes a perfume container for accumulating the perfume, arranges the perfume container at a position distant from the perfume presentation position at least while the perfume is not presented, and derives the perfume from the perfume container when the perfume in the perfume container is presented. The perfume conductor conducts the perfume derived by the perfume holder, to the perfume presentation module.

### CITATION LIST

### Patent Literature

[PTL 1]
PCT Patent Publication No. WO2016/199441

### SUMMARY

### Technical Problem

However, there is a possibility that the technology of PTL 1 may give, by presenting an unintended scent due to a remaining scent still attached to a casing, scent leakage, or the like, a sense of discomfort to the user in terms of the manner of sensing the scent different from that of a real world, and there is still room for improvement.

Therefore, it is a main object of the present technology to provide a scent provision apparatus capable of providing a scent to the user at a proper timing and preventing the provision of a scent at an unintended timing. Here, a “scent” indicates that all smells such as good and bad scents are included.

### Solution to Problem

The present technology provides a scent provision apparatus that includes a main body section and an air flow control unit. The main body section has an opening portion for discharging or taking in a scent-containing air flow that contains a scent. The air flow control unit controls characteristics of the scent-containing air flow discharged or taken in from the opening portion. The air flow control unit can generate a control air flow for accelerating and decelerating and/or diffusing the scent-containing air flow, and can also generate a control air flow for interrupting a flow of the scent-containing air flow.

The present technology also provides a scent provision method that includes a step of discharging or taking in a scent-containing air flow that contains a scent and a step of controlling characteristics of the scent-containing air flow discharged or taken in.

### Advantageous Effect of Invention

According to a scent provision apparatus according to the present technology, it is possible to provide a scent to a user at a proper timing and prevent the provision of a scent at an unintended timing. It should be noted that the above advantageous effect is not necessarily restrictive and that any one of the advantageous effects indicated in the present specification or other advantageous effects that can be grasped from the present specification may be produced together with or in place of the above advantageous effect.

### BRIEF DESCRIPTION OF DRAWINGS

FIG. 1 is a schematic configuration diagram illustrating a configuration example of a scent provision apparatus according to a first embodiment of the present technology.

FIG. 2 is a partial sectional diagram illustrating the configuration example of the scent provision apparatus according to the first embodiment of the present technology.

FIG. 3 is a schematic configuration diagram describing an operation example of the scent provision apparatus according to the first embodiment of the present technology.

FIG. 4 is a schematic configuration diagram illustrating a configuration example of a scent provision apparatus according to a second embodiment of the present technology.

FIG. 5 depicts schematic configuration diagrams each describing an operation example of the scent provision apparatus according to the second embodiment of the present technology.

FIG. 6 depicts schematic configuration diagrams each describing an operation example of the scent provision apparatus according to the second embodiment of the present technology.

FIG. 7 depicts schematic configuration diagrams each describing an operation example of the scent provision apparatus according to the second embodiment of the present technology.

FIG. 8 depicts schematic configuration diagrams each describing an operation example of the scent provision apparatus according to the second embodiment of the present technology.

FIG. 9 depicts schematic configuration diagrams each describing an operation example of the scent provision apparatus according to the second embodiment of the present technology.

FIG. 10 depicts schematic configuration diagrams each describing an operation example of the scent provision apparatus according to the second embodiment of the present technology.

### DESCRIPTION OF EMBODIMENTS

Preferred embodiments for carrying out the present technology will be described below with reference to drawings. The embodiments described below are typical examples of embodiments of the present technology, and any of the embodiments can be used in combination with another embodiment. Also, the scope of the present technology is not construed narrowly by these. It should be noted that the description will be given in the following order.
1. First Embodiment
(1) Configuration example of scent provision apparatus
(2) Operation example of scent provision apparatus
2. Second Embodiment
(1) Configuration example of scent provision apparatus
(2) Operation example 1 of scent provision apparatus
(3) Operation example 2 of scent provision apparatus
(4) Operation example 3 of scent provision apparatus
(5) Operation example 4 of scent provision apparatus
(6) Operation example 5 of scent provision apparatus
(7) Operation example 6 of scent provision apparatus
(8) Operation example 7 of scent provision apparatus

### 1. First Embodiment

(1) Configuration Example of Scent Provision Apparatus

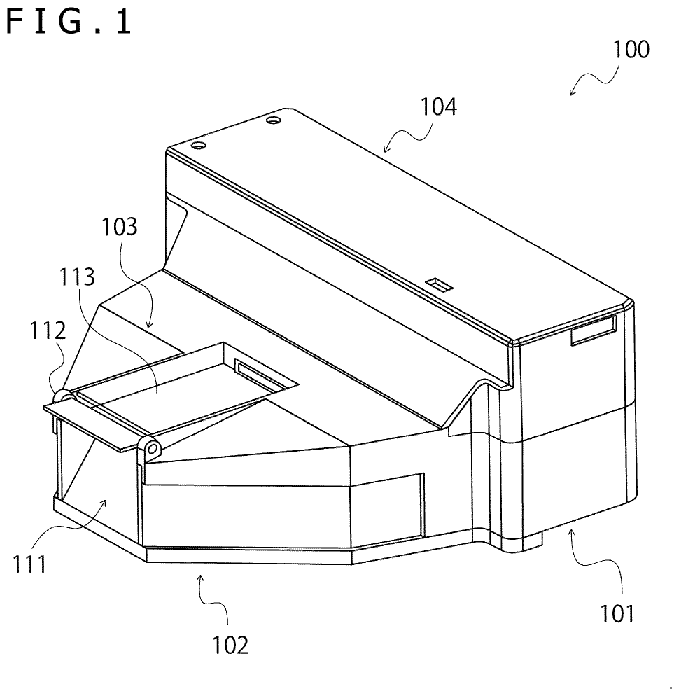

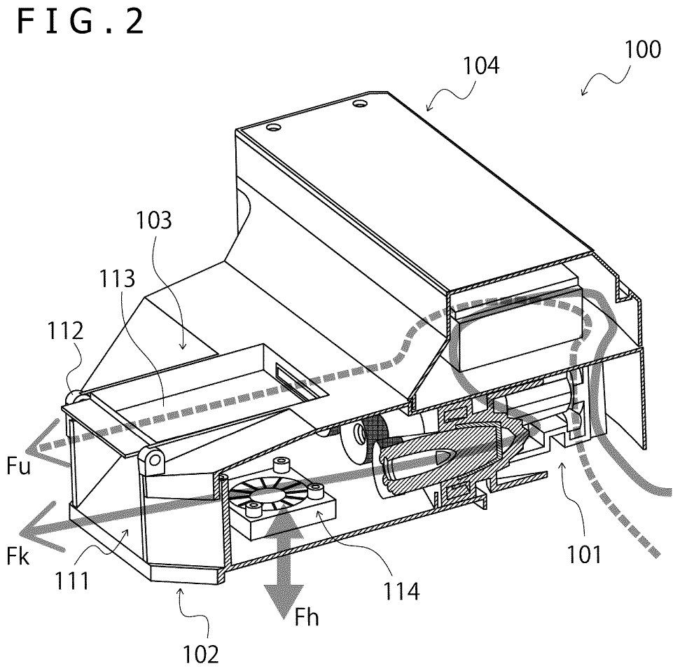

A description will be given of a configuration example of a scent provision apparatus 100 according to a first embodiment of the present technology with reference to FIGS. 1 and 2. FIG. 1 is a schematic configuration diagram illustrating the configuration example of the scent provision apparatus 100 according to the present embodiment. FIG. 2 is a partial sectional diagram illustrating the configuration example of the scent provision apparatus 100.

As illustrated in FIG. 1, the scent provision apparatus 100 such as an olfactory display includes a perfume holding unit 101, an air flow control unit 102, and a warm/cool control unit 104. The air flow control unit 102 is coupled to the perfume holding unit 101. The warm/cool control unit 104 is coupled to the perfume holding unit 101 and the air flow control unit 102.

The perfume holding unit 101 has, for example, a perfume holding member for holding a perfume which is a substance from which a scent to be provided to a user originates.

The air flow control unit 102 has an air flow control section 103. The air flow control section 103 has an opening portion 111, a flap 112, and an air flow path section 113. The opening portion 111 discharges externally or takes in a scent-containing air flow Fk that contains a scent. The flap 112 is arranged at a tip of the opening portion 111 and changes the flow of the scent-containing air flow Fk. The air flow path section 113 allows a control air flow Fu for controlling characteristics of the scent-containing air flow Fk, such as a direction and/or intensity, to flow therethrough. As illustrated in FIG. 2, the air flow control unit 102 has therein an air flow adjustment fan 114 that generates an adjustment air flow Fh for agitating or giving out an air flow. It should be noted that the air flow control unit 102 is preferably arranged near the opening portion 111. Also, a thermoelectric coil, a Peltier element, a water absorber (vaporization heat cooling), or the like may be integrally built into the air flow control unit 102.

The air flow control unit 102 can also generate the control air flow Fu for accelerating and decelerating and/or diffusing the scent-containing air flow Fk. It should be noted that “diffusing” includes the meaning that a perfume concentration in the air flow is reduced by blowing off the perfume and the perfume-containing air flow is spread therearound. Also, the air flow control unit 102 can generate the control air flow Fu for interrupting the flow of the scent-containing air flow as well. The air flow control unit 102 can also control the intensity of the scent-containing air flow Fk by mixing the plurality of control air flows Fu. The air flow control unit 102 can also take in and give out air from and to an outer periphery near the opening portion.

The warm/cool control unit 104 discharges or takes in a warm or cool air flow according to the scent included in the scent-containing air flow Fk. Also, the warm/cool control unit can adjust humidity of the scent-containing air flow Fk as well.

(2) Operation Example of Scent Provision Apparatus

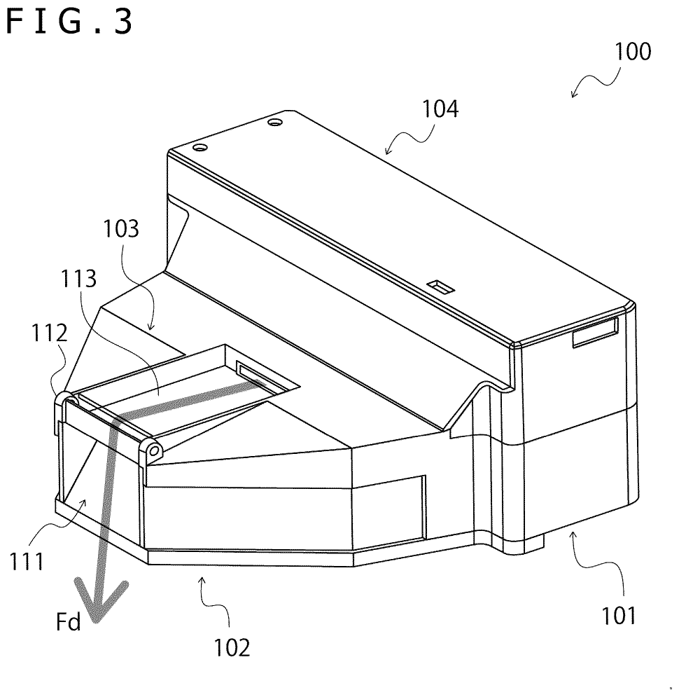

A description will be given next of an operation example for detecting a scent by the scent provision apparatus 100 with reference to FIGS. 1 to 3. FIG. 3 is a schematic configuration diagram describing an operation example of the scent provision apparatus 100. It should be noted that an operation procedure given below can be performed by changing an order.

As an example, in a first step, the perfume holding member is caused to hold the perfume of the scent to be provided to the user, and the perfume holding member holding the perfume is built into the perfume holding unit 101.

In a second step, as illustrated in FIG. 2, an air flow is flowed into the perfume holding unit 101 of the scent provision apparatus 100, and the flowed air flow is caused to flow into the warm/cool control unit 104 once. The air flow that has flowed into the warm/cool control unit 104 is warmed up or cooled down according to a circumstance in which the user uses the scent. Also, depending on the case, the humidity of the air flow is adjusted.

In a third step, the air flow flowed into the warm/cool control unit 104 is divided into two parts. One of them is flowed again into the perfume holding unit 101, and the other is flowed into the air flow path section 113 located above the air flow control unit 102.

In a fourth step, the air flow flowed into the perfume holding unit 101 is caused to flow near the perfume holding member, which creates the scent-containing air flow Fk. The scent-containing air flow Fk is flowed into the air flow control unit 102. Meanwhile, the air flow flowed into the air flow path section 113 is caused to flow above the opening portion 111 located at a tip of the air flow control unit 102, as the control air flow Fu for controlling the characteristics of the scent-containing air flow Fk.

In a fifth step, the scent-containing air flow Fk flowed into the air flow control unit 102 is agitated by the adjustment air flow Fh generated from the air flow adjustment fan 114 or given out, and then discharged externally or taken in from the opening portion 111.

In a sixth step, the characteristics of the scent-containing air flow Fk are controlled by adjusting the intensity of the control air flow Fu caused to flow above the opening portion 111 and the direction thereof by the flap 112.

As an example of control over the characteristics of the scent-containing air flow Fk, the scent provision apparatus 100 can cause the control air flow Fu to flow from above to below the opening portion 111 as a descending air flow Fd by moving the flap 112, which is arranged at a tip of the air flow path section 113, to a downward position, as illustrated in FIG. 3. This makes it possible to create an air curtain in the opening portion 111 generated by the descending air flow Fd and stop the scent-containing air flow Fk from being discharged externally from the scent provision apparatus 100.

As described above, the scent provision apparatus 100 according to the present embodiment has the air flow control unit 102 near the tip opening portion 111, which makes it possible to control directivity and intensity of the air flow at a root of the user's nose by collision or confluence of the scent-containing air flow Fk and the control air flow Fu. This enables the scent provision apparatus 100 to imitate a natural wafting scent by causing the user to perceive the scent-containing air flow Fk and sense the scent together with a wind or, conversely, weakening the air flow at the root of the nose and making it difficult for the user to perceive the scent.

Also, the scent provision apparatus 100 can prevent perception of an unintended smell such as a remaining scent or smell leakage by generating an air flow or an air shutter to be inhaled near the opening portion 111, when the user is not desired to perceive a smell at a time other than at the time of presentation of a perfume. In consequence, according to the scent provision apparatus 100, it is possible to provide a scent to the user at a proper timing and prevent the provision of a scent at an unintended timing.

It should be noted that, although, in the present embodiment, the warm/cool control unit 104 is provided in the scent provision apparatus 100, the warm/cool control unit 104 may not be provided, and instead, a biological activity detection unit for detecting a biological activity can be provided. Also, the scent provision apparatus 100 not only can provide a scent but also can have a function as a deodorant by taking in an air flow around the user. Further, the scent provision apparatus 100 can prevent a user's airborne droplet from flowing into and adhering to the scent provision apparatus 100, by an air shutter generated by an air flow.

### 2. Second Embodiment

(1) Configuration Example of the Scent Provision Apparatus

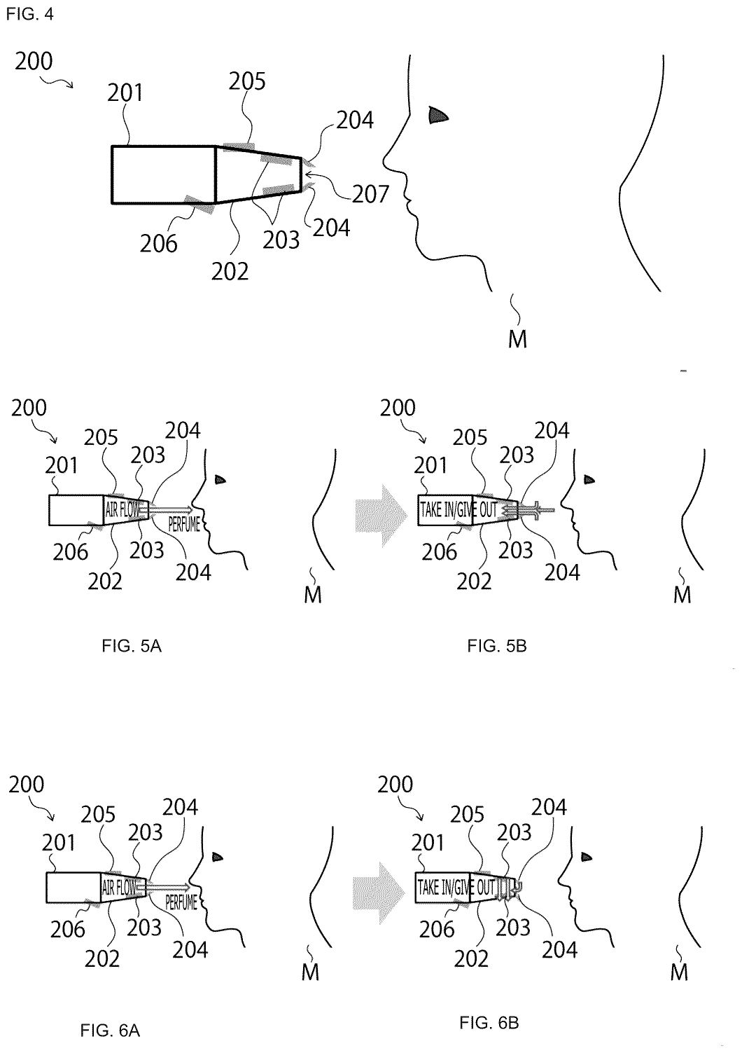

A description will be given next of a configuration example of a scent provision apparatus 200 according to a second embodiment of the present technology with reference to FIG. 4. FIG. 4 is a schematic configuration diagram illustrating a configuration example of the scent provision apparatus 200. The scent provision apparatus 200 is more compact than the scent provision apparatus 100 according to the first embodiment and can provide a scent to a face of a user M, and especially, at a position near the vicinity of the nose.

As illustrated in FIG. 4, the scent provision apparatus 200 according to the present embodiment includes a main body section 201, a nose cover section 202, and an air flow control unit 203. The nose cover section 202 is attached to the main body section 201. Further, the scent provision apparatus 200 includes a flap 204, a heartbeat-posture detection unit 205, and a respiration detection unit 206. The scent provision apparatus 200 has, at a tip of the nose cover section 202, an opening portion 207 for taking in and giving out an air flow.

The main body section 201 can include a perfume holding member for holding a perfume and controls overall operation of the scent provision apparatus 200. In addition, the main body section 201 also has an information acquisition section for acquiring biological information of the user M, such as vital information and posture information.

The nose cover section 202 is arranged at a tip of the main body section 201 and has a function of gathering scents generated by the scent-containing air flow discharged from the main body section 201.

The air flow control unit 203 is arranged at the tip of the nose cover section 202. As a specific example of the air flow control unit 203, an axial fan, a centrifugal fan, a sirocco fan, a diaphragm air pump, a linear air pump, a compression pump, a chemical reaction type gas generator, and the like can be used.

The flap 204 is attached near the opening portion 207 formed at the tip of the nose cover section 202. As the flap 204, for example, an air flow control variable multi-flap, a louver, and the like that control an air flow can be used.

The heartbeat-posture detection unit 205 is, as an example, arranged on the nose cover section 202 and has a sensor for detecting the heartbeat and posture. Also, the respiration detection unit 206 is, as an example, arranged on the main body section 201 and has a sensor for detecting the respiration of the user M. As specific examples of the heartbeat-posture detection unit 205 and the respiration detection unit 206, a TOF, infrared rays, an image sensor, a microwave radar, a millimeter wave radar, and the like can be used.

The heartbeat-posture detection unit 205 and the respiration detection unit 206 can be included in the scent provision apparatus 200 as a biological activity detection unit for detecting a biological activity of the user M. The scent provision apparatus 200 controls the scent-containing air flow in conjunction with an action of the biological activity detected by the biological activity detection unit. Here, the biological activity refers to an activity selected from a group of respiration, posture, and heartbeat of the user M and combinations thereof. It should be noted that the posture includes not only the position and orientation of the face of the user M but also the position and orientation of the nose.

(2) Operation Example 1 of the Scent Provision Apparatus

A description will be given next of an operation example 1 of scent provision by the scent provision apparatus 200 with reference to FIG. 5. FIG. 5 depicts schematic configuration diagrams each describing an operation example of presentation of an air flow and taking in and giving out of an air flow by the scent provision apparatus 200. FIG. 5A illustrates a timing when the user M is desired to sense the scent, and FIG. 5B illustrates a timing when the user M is not desired to sense the scent.

As illustrated in FIG. 5A, in the case where the user M is desired to sense the scent, the scent-containing air flow generated by the main body section 201 is caused to flow into the nose cover section 202 and discharged externally from the opening portion 207. At this time, the scent provision apparatus 200 provides a perfume by causing the air flow control unit 203 to generate a control air flow appropriate to the circumstance and ejecting a scent-containing air flow controlled by the control air flow, toward the vicinity of the nose of the user M.

As illustrated in FIG. 5B, in the case where the user M is not desired to sense the scent, the scent-containing air flow is controlled by taking in the scent-containing air flow from the opening portion 207 by the control air flow or giving out the scent-containing air flow from the main body section 201 so as not to cause wafting of a remaining scent or scent leakage. It should be noted that the scent provision apparatus 200 can also control the scent-containing air flow in such a manner as to cause the scent to waft.

(3) Operation Example 2 of Scent Provision Apparatus

A description will be given next of an operation example 2 of scent provision by the scent provision apparatus 200 with reference to FIG. 6. FIG. 6 depicts schematic configuration diagrams each describing an operation example of presentation and containment of an air flow by the scent provision apparatus 200. FIG. 6A illustrates a timing when the user M is desired to sense the scent, and FIG. 6B illustrates a timing when the user M is not desired to sense the scent.

As illustrated in FIG. 6A, in the case where the user M is desired to sense the scent, a perfume is provided by causing the air flow control unit 203 to generate a control air flow appropriate to the circumstance and ejecting a scent-containing air flow controlled by the control air flow, toward the vicinity of the nose of the user M, as in the above operation example 1.

As illustrated in FIG. 6B, in the case where the user M is not desired to sense the scent, for example, the scent-containing air flow is controlled so as not to cause wafting of a remaining scent or scent leakage, by generating an air shutter in the opening portion 207 by the control air flow flowing from above the nose cover section 202 to therebelow.

(4) Operation Example 3 of Scent Provision Apparatus

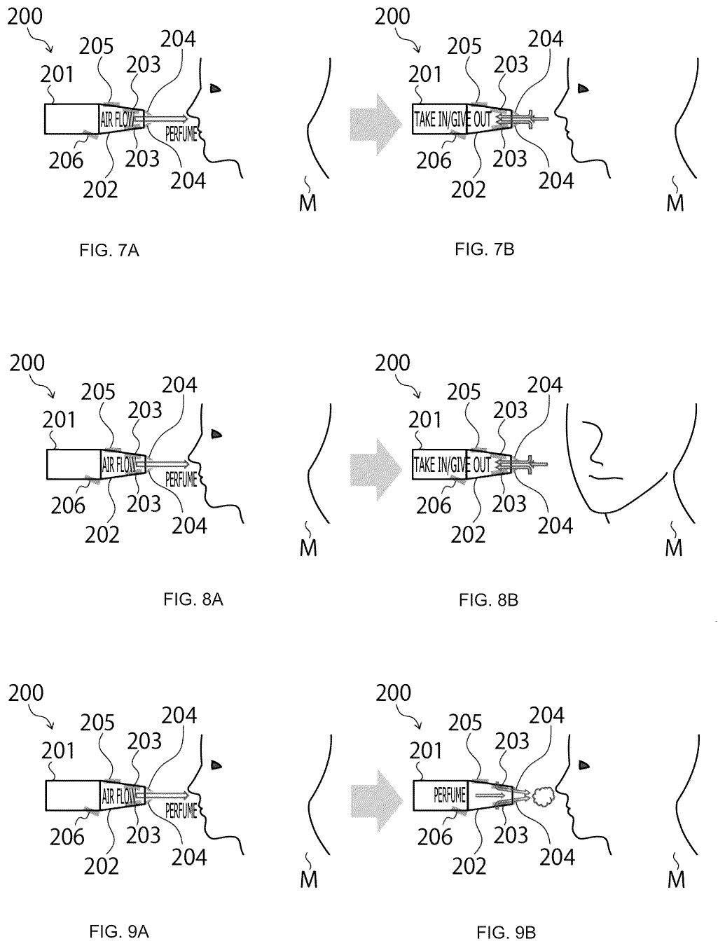

A description will be given next of an operation example 3 of scent provision by the scent provision apparatus 200 with reference to FIG. 7. FIG. 7 depicts schematic configuration diagrams each describing an operation example of air flow control through respiration detection by the scent provision apparatus 200. FIG. 7A illustrates a case where the user M breathes in, and FIG. 7B illustrates a case where the user M breathes out.

As illustrated in FIG. 7A, in the case where the user M breathes in, a breathing-in action is detected by the respiration detection unit 206, and a perfume generated by a scent-containing air flow controlled by a control air flow generated to suit the detected action is provided to the user M.

As illustrated in FIG. 7B, in the case where the user M breathes out, a breathing-out action is detected by the respiration detection unit 206, and it is possible to prevent wafting of a remaining scent or scent leakage and avoid an unintended perfume, by taking in or giving out a scent-containing air flow by the control air flow generated to suit the detected action.

(5) Operation Example 4 of Scent Provision Apparatus

A description will be given next of an operation example 4 of scent provision by the scent provision apparatus 200 with reference to FIG. 7. FIG. 7 depicts schematic configuration diagrams each describing an operation example of air flow control through heartbeat detection by the scent provision apparatus 200. FIG. 7A illustrates a case where a perfume is desired, in reference to the heartbeat of the user M, to be presented, and FIG. 7B illustrates a case where a perfume is not desired, in reference to the heartbeat of the user M, to be presented.

As illustrated in FIG. 7A, in the case where a perfume is desired, in reference to the heartbeat of the user M, to be presented, a heart rate of the user M is detected by the heartbeat-posture detection unit 205. If the heart rate is such that a perfume is desired to be presented, a control air flow is generated by the air flow control unit 203 to suit the heart rate, and a perfume generated by a scent-containing air flow controlled by the control air flow is provided to the user M.

As illustrated in FIG. 7B, in the case where a perfume is not desired, in reference to the heartbeat of the user M, to be presented, the heart rate of the user M is also detected by the heartbeat-posture detection unit 205. If the heart rate is such that a perfume is not desired to be presented, it is possible to prevent wafting of a remaining scent or scent leakage and avoid an unintended perfume, by taking in or giving out a scent-containing air flow by the control air flow generated by the air flow control unit 203 to suit the heart rate.

(6) Operation Example 5 of Scent Provision Apparatus

A description will be given next of an operation example 5 of scent provision by the scent provision apparatus 200 with reference to FIG. 8. FIG. 8 depicts schematic configuration diagrams each describing an operation example of air flow control through posture detection by the scent provision apparatus 200. FIG. 8A illustrates a case where a perfume is desired, in reference to the posture of the user M, to be presented, and FIG. 8B illustrates a case where a perfume is not desired, in reference to the posture of the user M, to be presented.

As illustrated in FIG. 8A, in the case where a perfume is desired, in reference to the posture of the user M, to be presented, the posture of the user M is detected by the heartbeat-posture detection unit 205. If the posture is such that a perfume is desired to be presented, a control air flow is generated by the air flow control unit 203 to suit the posture, and a perfume generated by the scent-containing air flow controlled by the control air flow is provided to the user M. In FIG. 8A, the posture at the time when the user M brings the vicinity of his or her nose close to the scent provision apparatus 200 is the posture in the case where a perfume is desired to be presented.

As illustrated in FIG. 8B, also in the case where a perfume is not desired, in reference to the posture of the user M, to be presented, the posture of the user M is detected by the heartbeat-posture detection unit 205. If the posture is such that a perfume is not desired to be presented, it is possible to prevent wafting of a remaining scent or scent leakage and avoid an unintended perfume, by taking in or giving out a scent-containing air flow by the control air flow generated by the air flow control unit 203 to suit the posture. In FIG. 8B, the posture at the time when the user M turns the vicinity of his or her nose away from the scent provision apparatus 200 is the posture in the case where a perfume is not desired to be presented.

(7) Operation Example 6 of Scent Provision Apparatus

A description will be given next of an operation example 6 of scent provision by the scent provision apparatus 200 with reference to FIG. 9. FIG. 9 depicts schematic configuration diagrams each describing an operation example of air flow control for calming a perfumed air flow by the scent provision apparatus 200. FIG. 9A illustrates a case where the user M desires to cause a wind and a scent to be sensed, and FIG. 9B illustrates a case where the user M desires to gently sense a scent but does not desire to sense a wind.

As illustrated in FIG. 9A, in the case where the user M desires to cause a wind and a scent to be sensed, a perfume is provided by generating a control air flow appropriate to the circumstance with the air flow control unit 203 and ejecting a scent-containing air flow controlled by the control air flow, toward the vicinity of the nose of the user M, as in the above operation example 1.

As illustrated in FIG. 9B, in the case where the user M desires to gently sense a scent but does not desire to sense a wind, the scent-containing air flow flowing from the main body section 201 and the control air flow generated by the air flow control unit 203 are caused to collide near the root of the nose of the user M. This makes it possible for the scent provision apparatus 200 to diffuse the scent-containing air flow and cause the scent-containing air flow to be scented gently near the root of the nose without sensing a wind.

(8) Operation Example 7 of Scent Provision Apparatus

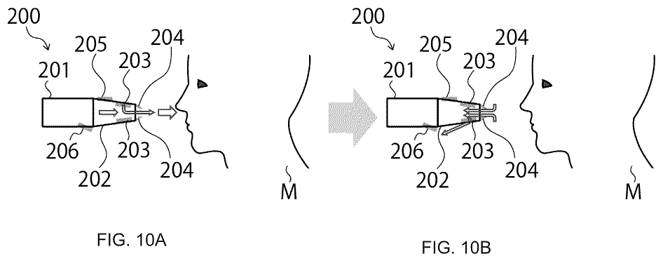

A description will be given next of an operation example 7 of scent provision by the scent provision apparatus 200 with reference to FIG. 10. FIG. 10 depicts schematic configuration diagrams each describing an operation example of selective use of fans for presentation and giving out of a scent by the scent provision apparatus 200. FIG. 10A illustrates a scent presentation mode (presentation side ON), and FIG. 10B illustrates a scent giving-out mode (giving-out side ON). In this case, a plurality of fans is included in the air flow control unit 203.

As illustrated in FIG. 10A, in the case of the scent presentation mode, a control air flow for presenting a scent is generated by turning ON a scent presentation fan for presenting a scent to the user M. The scent provision apparatus 200 provides the scent to the user M by controlling a scent-containing air flow by the generated control air flow.

As illustrated in FIG. 10B, in the case of the scent giving-out mode, a control air flow for giving out a scent is generated by turning ON a scent giving-out fan for giving out a scent-containing air flow in a direction of moving it further away or turning it away from the vicinity of the nose of the user M, in order to exclude a remaining scent or scent leakage when the scent is not presented. The scent provision apparatus 200 blows and gives out all air present near the nose and near the tip opening portion 207 in the direction opposite to the nose by the generated control air flow.

It should be noted that the scent provision apparatus 200 can also take in and give out a scent-containing air flow constantly from near the nose cover section 202 in a background for the purpose of preventing scent leakage and drifting of material smells of the nose cover section 202 and other sections, for example, before the scent is provided to the user.

As described above, according to the scent provision apparatus 200 according to the present embodiment, it is possible to provide a scent to the user M at a proper timing and prevent the provision of a scent at an unintended timing, as with the scent provision apparatus 100 according to the first embodiment. Further, the scent provision apparatus 200 has the biological activity detection unit that includes the heartbeat-posture detection unit 205 and the respiration detection unit 206, which makes it possible to acquire biological activity information such as respiration, heartbeat, and face orientation of the user M and generate a scent-containing air flow near the opening portion 207 to suit these signals at a timing when the user M is desired to smell the scent-containing air flow.

The scent provision apparatus according to the present technology can be used, for example, for an olfactory display for artificially presenting a scent together with a video image and other apparatuses. Further, the scent provision apparatus according to the present technology can also be used for a testing apparatus for measuring olfactory capability, for example.

It should be noted that the present technology can have the following configuration.
(1)

A scent provision apparatus including:
a main body section having an opening portion for discharging or taking in a scent-containing air flow that contains a scent; and
an air flow control unit adapted to control characteristics of the scent-containing air flow discharged or taken in from the opening portion.
(2)

The scent provision apparatus according to (1), in which
the air flow control unit generates a control air flow for accelerating and decelerating and/or diffusing the scent-containing air flow.
(3)

The scent provision apparatus according to (1) or (2), in which
the air flow control unit generates a control air flow for interrupting a flow of the scent-containing air flow.
(4)

The scent provision apparatus according to any one of (1) to (3), in which
the air flow control unit controls intensity of the scent-containing air flow by mixing a plurality of control air flows.
(5)

The scent provision apparatus according to any one of (1) to (4), in which
the air flow control unit takes in and gives out air from and to an outer periphery near the opening portion.
(6)

The scent provision apparatus according to any one of (1) to (5), in which
the air flow control unit has, at a tip of the opening portion, a flap for changing a flow of the scent-containing air flow.
(7)

The scent provision apparatus according to any one of (1) to (6), further including:
a biological activity detection unit adapted to detect a biological activity of a user, in which
the scent-containing air flow is controlled in conjunction with an action of the biological activity.
(8)

The scent provision apparatus according to (7), in which
the biological activity is an activity selected from a group of respiration, posture, and heartbeat of the user and combinations thereof.
(9)

The scent provision apparatus according to any one of (1) to (8), in which
the main body section has a nose cover section that has the opening portion, and
the air flow control unit is arranged near the opening portion.
(10)

The scent provision apparatus according to any one of (1) to (9), further including:
an information acquisition section adapted to acquire biological information of the user.
(11)

The scent provision apparatus according to any one of (1) to (10), further including:
a warm/cool control unit adapted to discharge or take in a warm or cool air flow according to the scent included in the scent-containing air flow.
(12)

The scent provision apparatus according to (11), in which
the warm/cool control unit adjusts humidity of the scent-containing air flow.
(13)

A scent provision method including:
a step of discharging or taking in a scent-containing air flow that contains a scent originated from the perfume; and
a step of controlling characteristics of the scent-containing air flow discharged or taken in.

### REFERENCE SIGNS LIST

100, 200: Scent provision apparatus
101: Perfume holding unit
102, 203: Air flow control unit
103: Air flow control section
104: Warm/cool control unit
111: Opening portion
112, 204: Flap
113: Air flow path section
114: Air flow adjustment fan
201: Main body section
202: Nose cover section
205: Heartbeat-posture detection unit
206: Respiration detection unit
207: Opening portion

1. A scent provision apparatus comprising:
a main body section having an opening portion for discharging or taking in a scent-containing air flow that contains a scent; and
an air flow control unit adapted to control characteristics of the scent-containing air flow discharged or taken in from the opening portion.

2. The scent provision apparatus according to claim 1, wherein
the air flow control unit generates a control air flow for accelerating and decelerating and/or diffusing the scent-containing air flow.

3. The scent provision apparatus according to claim 1, wherein
the air flow control unit generates a control air flow for interrupting a flow of the scent-containing air flow.

4. The scent provision apparatus according to claim 1, wherein
the air flow control unit controls intensity of the scent-containing air flow by mixing a plurality of control air flows.

5. The scent provision apparatus according to claim 1, wherein
the air flow control unit takes in and gives out air from and to an outer periphery near the opening portion.

6. The scent provision apparatus according to claim 1, wherein
the air flow control unit has, at a tip of the opening portion, a flap for changing a flow of the scent-containing air flow.

7. The scent provision apparatus according to claim 1, further comprising:
a biological activity detection unit adapted to detect a biological activity of a user, wherein
the scent-containing air flow is controlled in conjunction with an action of the biological activity.

8. The scent provision apparatus according to claim 7, wherein
the biological activity is an activity selected from a group of respiration, posture, and heartbeat of the user and combinations thereof.

9. The scent provision apparatus according to claim 1, wherein
the main body section has a nose cover section that has the opening portion, and
the air flow control unit is arranged near the opening portion.

10. The scent provision apparatus according to claim 1, further comprising:
an information acquisition section adapted to acquire biological information of the user.

11. The scent provision apparatus according to claim 1, further comprising:
a warm/cool control unit adapted to discharge or take in a warm or cool air flow according to the scent included in the scent-containing air flow.

12. The scent provision apparatus according to claim 11, wherein
the warm/cool control unit adjusts humidity of the scent-containing air flow.

13. A scent provision method comprising:
a step of discharging or taking in a scent-containing air flow that contains a scent; and
a step of controlling characteristics of the scent-containing air flow discharged or taken in.
